(19) United States
(12) Patent Application Publication
(10) Pub. No.: US 2005/0266475 A1
(43) Pub. Date: Dec. 1, 2005

(54) Method for determining an analyte

(75) Inventors: Patrick Chaton, Theys (FR); Francoise Vinet, Grenoble (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: COMMISSARIAT A L'ENERGIE ATOMIQUE, Paris (FR)

(21) Appl. No.: 11/143,997

(22) Filed: Jun. 3, 2005

Related U.S. Application Data
(62) Division of application No. 10/450,158, filed on Jun. 11, 2003, now abandoned.

(30) Foreign Application Priority Data
Dec. 20, 2000 (FR) 00/16650

(51) Int. Cl. C12Q 1/68; C12M 1/34

(52) U.S. Cl. 435/6; 435/287.2

(57) ABSTRACT

The invention relates to an analytical support for determining an analyte such as a DNA or RNA target, by carrying out a specific reaction of binding (hybridization) of said analyte with a ligand specific for this analyte, and determining the binding reaction by means of at least two fluorescent labels present on the analyte. This determination is carried out by applying an excitatory field and detecting the fluorescent emission of the various fluorescent labels. According to the invention, the support comprises a substrate coated with one or more layers of material(s) forming an assembly capable of decreasing or increasing the excitatory field for at least one of the fluorescent labels compared to the others. The ligands are attached to the final layer of the assembly.

A layer of SiO₂ on a substrate of silicon can be used with the fluorescent labels Cy3 and Cy5.

### DESCRIPTION

1. Technical Field

The present invention relates to an analytical support or “biochip” intended for determining analytes such as DNA or RNA targets, proteins, antigens and antibodies.

More precisely, it relates to biochips the principle of which is based on the detection of a specific reaction of binding of the analyte with a ligand specific for this analyte and detection of this reaction by means of fluorescent labels.

These biochips find applications in many fields, in particular in biology, for sequencing genomes, searching for mutations, developing novel medicinal products, etc.

2. State of the Art

When the analyte is a biological target of the DNA or RNA type, an analytical support of this type comprises a plurality of oligonucleotide probes capable of giving rise to a hybridization with the biological targets to be analysed. The hydridization corresponds to the pairing of the target-strands with the complementary DNA strands arranged on the support. To determine the nature of the targets, it is therefore necessary to be able to locate which sites of the support and therefore which oligonucleotides have given rise to a hydridization.

The document Médecine/Sciences, Vol. 13, No. 11, 1997, pp. 1317-1324 [1] describes analytical supports of this type.

Conventionally, the hybridization of the biological targets on the support is determined using a fluorescent label which is associated with the biological targets, after extraction of the regions of interest (lysis and optional amplification).

After the labelled targets have been brought into contact with the analytical support comprising the oligoprobes, the sites where the hybridization takes place are determined by exciting all of the fluorescent labels and then reading the sites in order to detect the fluorescent light re-emitted by the labels. The sites for which fluorescent light is detected are those which have bound the target molecules.

The detection is carried out using confocal microscopes or scanners of the “General Scanning” or “Genetic Microsystem” type. Reading systems suitable for various biochips are described, for example, in U.S. Pat. No. 5,578,832 [2] and U.S. Pat. No. 5,646,411 [3].

This principle of detection of the targets by fluorescent labelling is sound and simple to implement; moreover, it exhibits an ultimate sensitivity of detection since single targets can be demonstrated without extensive instrumentation.

Generally, a single fluorescent label which is associated with the biological targets is used for this detection. However, it would be advantageous to simultaneously use several fluorescent labels since this redundancy makes it possible to unambiguously detect the biological target, which is particularly advantageous for studying the gene expression and confirming the signature of the gene studied.

However, when two fluorescent labels are used, one of the labels may have a greater luminescence yield and, by the same token, it may be incompatible with a second fluorescent label of weaker intensity. A correction of the level of fluorescence of the most intense label is difficult to carry out since it acts directly on the density of the targets and is, as a result, difficult to control.

Moreover, the reader scanners exhibit spectral selectivity between fluorophores, which can distort the analysis of the signals from the biochip. Modification of the scanners is difficult for the non-specialist since they are mostly commercial devices and adapting them to chosen fluorescent labels is an approach which can prove to be very laborious.

### EXPLANATION OF THE INVENTION

A subject of the present invention is precisely an analytical support designed so as to compensate for the difference in luminescence between several fluorescent labels, which can, in addition, adapt to the spectral responses of reading scanners.

Thus, a subject of the invention is an analytical support for determining an analyte by carrying out a specific reaction of binding of said analyte with a ligand specific for this analyte, and determining the binding reaction by means of at least two fluorescent labels present on the analyte, this determination being carried out by applying an excitatory field and detecting the fluorescent emission of the various fluorescent labels, said support comprising a substrate to which is attached a plurality of ligands specific for the analyte to be determined, said substrate being coated with one or more layer(s) of material(s) forming an assembly capable of decreasing or increasing the excitatory field for at least one of the fluorescent labels compared to the others, and the ligands being attached to the final layer of the assembly.

It is specified that the term “analyte” is intended to mean any chemical or biochemical compound capable of producing a reaction consisting of binding with a ligand specific for this analyte. By way of example of an analyte, mention may be made of biological targets such as oligonucleotides, namely DNA or RNA probes, proteins and their receptors, and immunological compounds of the antigen and antibody type.

The analyte/analyte-specific ligand pairs can be chosen from the following pairs: oligonucleotide-complementary oligonucleotide, antibody-antigen, protein-protein receptor, or vice versa.

According to the invention, the substrate of the analytical support is coated with one or more layers made of one or more materials forming an assembly capable of decreasing or increasing the excitatory field for at least one of the fluorescent labels. The layer(s) form an assembly which plays the role of an optical filter which exhibits characteristics of optical filtering while at the same time being transparent to the excitation and emission wavelengths of the fluorescent labels.

The nature of the material(s) constituting said layer(s), and also the thickness of the layer(s), should therefore be chosen so as to obtain this decrease or this increase in the excitatory field for at least one of the fluorescent labels compared to the others.

The thickness of each layer is chosen as a function of the refractive index n of the material constituting it, at the wavelength λ of maximum absorption of the fluorescent label.

Thus, when a single thin layer is used, the thickness e of this layer should satisfy the following rule:
en₁=kλ_{M1}/4
where n₁ is the refractive index of the material at the wavelengths λ_{M1} of maximum absorption of the fluorescent label M₁, and k is an odd integer, so as to increase the excitation of the fluorescent label M1.

When several thin layers are used, the desired thicknesses for these thin layers can be calculated according to the teaching of the document Edward H. Hellen and Daniel Axelrod, J. Opt. Soc. Am. B, Vol. 4, No. 3, March 1987, p. 337 to 350 [4].

The thicknesses are of the order of magnitude of the excitation or emission wavelength of the fluorescent labels used.

The thin layer(s) has (have) a high optical quality and it (they) exhibit(s) characteristics of optical filtering.

The materials of the thin layers may be chosen from refractory oxides, metal fluorides and silicon oxynitrides.

By way of example of a refractory oxide, mention may be made of TiO₂, HfO₂, Ta₂O₅, SiO₂ and ZrO₂.

By way of example of a fluoride, mention may be made of YF₃ and MgF₂.

The material of the final layer to which the ligands specific for the analyte to be determined will be attached is chosen so as to allow this attachment.

The material of the first layer, which is directly in contact with the substrate, is chosen so as to be compatible with the substrate.

The substrates used may be of diverse type. In general, they are made of a semi-conductor, of glass, or of plastic material.

When a silicon substrate is used, the layer for decreasing or increasing the excitatory field is advantageously made of silica SiO₂.

Of course, the material(s) forming the layer(s) is (are) chosen as a function of the fluorescent labels used, so as to allow this decrease or this increase and to provide the equilibration of the signals emitted by these labels.

According to a preferred embodiment of the invention, the support is suitable for determining the signals emitted by two fluorescent labels.

For this purpose, various types of label can be used, for example the first fluorescent label may be Cy3, the excitation wavelength of which is approximately 550 nm and the emission wavelength of which is approximately 580 nm, and the second fluorescent label may be Cy5, the excitation wavelength of which is approximately 650 nm and the emission wavelength of which is approximately 680 nm.

With these labels, it is possible to use a layer of thermal silica having a thickness of approximately 100 to 1 000 nm, on a substrate made of silicon.

The signals emitted by the labels can be determined either simultaneously or successively.

A subject of the present invention is also a method of producing an analytical support exhibiting the characteristics given above.

This method consists:
1) in forming, on a substrate, one or more thin layers of material(s) intended to form the assembly capable of decreasing or increasing the excitatory field for at least one of the fluorescent labels, and
2) in attaching to the final layer formed on the substrate the ligands required for determining the analyte.

In general, the layer(s) of material(s) can be formed on the substrate by conventional techniques for producing optical thin layers, such as sol-gel deposition, chemical vapour deposition (CVD), physical vapour deposition (PVD), electron gun deposition, sputtering, or else thermal oxidation of the substrate.

When the ligands required for the determination are oligonucleotides, these oligonucleotides can be attached to the final layer formed on the support, by the techniques conventionally used to produce biochips.

These techniques can make use of piezoelectric dispensers which deposit, on the substrate, drops which contain the oligonucleotide and which are a few hundred micrometers in diameter, at the desired sites. Use may also be made of techniques based on the use of conductive polymers which immobilize the probes by copolymerization, as described in WO 94/22889 [5], or techniques comprising in situ synthesis of the oligonucleotide, in which the probes are extended base after base on the final layer formed on the substrate.

The in situ synthesis involves conventional reactions of coupling via phosphoramidites, phosphites or phosphonates, for the successive condensation of astutely protected nucleotides. The synthetic cycle comprises deprotection, coupling, blocking and oxidation steps, and makes it possible to extend the oligonucleotide from the surface of each site.

Preferably, prior treatment of the final layer is carried out in order to provide attachment of the ligands via spacer arms and thus to distance them from the surface of the support. This treatment may consist in grafting a spacer arm onto the final layer.

When the final layer is made of silica SiO₂, this treatment may consist of functionalization with epoxy groups, followed by a reaction with a glycol.

Other characteristics and advantages of the invention will become more clearly apparent on reading the following description of examples of implementation given, of course, by way of nonlimiting illustration, with reference to the attached drawings.

### BRIEF DESCRIPTION OF THE DRAWINGS

FIG. 1 is a diagram illustrating the absorption spectra of two fluorescent labels used in the invention;

FIG. 2 is a diagram illustrating the evolution of the excitatory fields as a function of the thickness (in nm) of the layer deposited onto the substrate for the fluorescent labels Cy3 and Cy5;

FIG. 3 is a diagram illustrating the evolution of the fluorescence intensity of the fluorescent labels Cy3 and Cy5 as a function of the thickness (in nanometres) of the layer of oxide SiO₂ present on the substrate; and

FIG. 4 represents the evolution of the properties of reflexion (in %) of a multilayer structure in accordance with the invention, as a function of the wavelength.

### DETAILED DESCRIPTION OF THE EMBODIMENTS

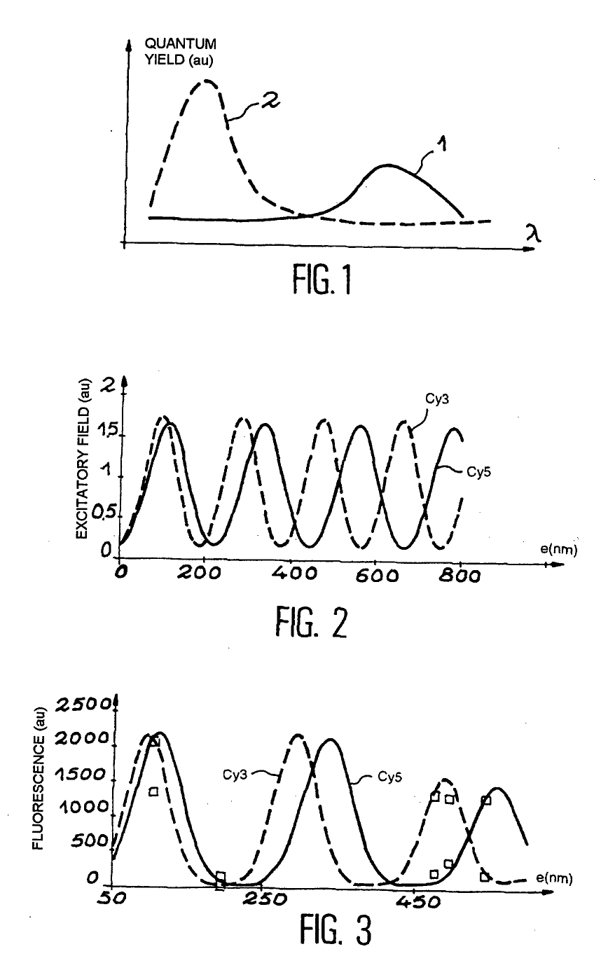

In FIG. 1, curve 1 represents the absorption spectrum of the first fluorescent label Cy3 used in the invention, and curve 2 represents the absorption spectrum of the second fluorescent label Cy5 used in the invention, namely the quantum yields as a function of the excitation wavelength.

In this figure, it is seen that the spectra of the two labels do not have the same amplitude. Thus, to obtain an advantageous response with these two labels, it is advisable either to decrease the excitatory field of the second label or to increase the excitatory field of the first label, in order to equilibrate the two signals emitted by the labels and to obtain a satisfactory response.

### EXAMPLE 1

In this example, a biochip is used which comprises a substrate made of silicon on which a single layer of thermal silica SiO₂ is formed. An electromagnetic calculation performed in a similar way to the calculation described in document [4] makes it possible to determine the optimal excitation thicknesses for the two fluorescent labels used, Cy3 and Cy5, which have the following characteristics:
Cy3: maximum wavelength of absorption equal to 550 nm and emission wavelength equal to approximately 580 nm;
Cy5: maximum wavelength of absorption equal to 650 nm and emission wavelength equal to approximately 680 nm.

FIG. 2 represents the calculated evolution of the excitatory fields as a function of the thickness (in nanometres) of the layer of thermal silica SiO₂, when the excitation is performed at the maximum wavelength of absorption of the two labels. In this figure, it is noted that there exist thicknesses of silica which make it possible to decrease the fluorescence of Cy3 compared to that of Cy5, and vice versa.

Based on these calculations, several analytical supports are produced with different thicknesses of thermal silica.

For this purpose, 10 substrates made with silicon are used, on which a layer of silica is formed by thermal oxidation in order to obtain substrates having the following thicknesses of SiO₂:
2 substrates with a layer of 100 nm,
2 substrates with a layer of 190 nm,
2 substrates with a layer of 475 nm,
2 substrates with a layer of 500 nm, and
2 substrates with a layer of 545 nm.

The substrates are then subjected to a cleaning by immersion in an alkaline solution of sodium hydroxide, and are rinsed in deionized water.

Next, a spacer arm is attached to the layer of SiO₂ by carrying out the following steps:
silanization of the substrates using glycidyloxypropyltrimethoxysilane; and
grafting of the spacer arm consisting of tetraethylene glycol.

These steps correspond to the following reactions:

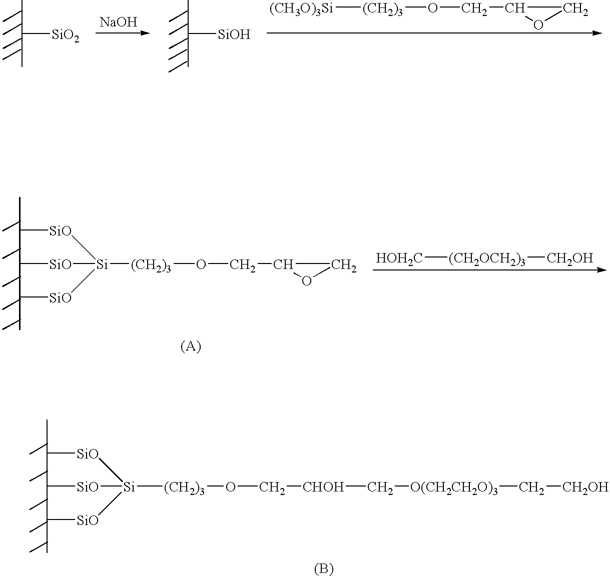

Thus, the substrates are first of all subjected to silanization by immersing them in a solution comprising 1 ml of glycidyloxypropyltrimethoxysilane in 3.5 ml of toluene and 0.3 ml of triethylamine overnight at 800° C. The substrates are then rinsed with acetone, dried, and re-cured at 110° C. for 3 hours.

The silanized substrates (A) are thus obtained. A spacer arm is then grafted onto the epoxy groups in order to distance the oligonucleotide from the surface and to thus promote the conditions for hybridization.

For this purpose, the substrates are treated in acid medium in order to catalyse the grafting reaction the spacer arm consisting of tetraethylene glycol (TEG). The treated substrate (B) is thus obtained.

After grafting of the spacer arm, the substrates are introduced into an Expedite 8909 automatic synthesizer in order to produce a programmed oligonucleotide sequence using phosphoramidite chemistry. 20-mer oligonucleotide probes comprising the following succession of bases:

3′, TTTTT ATC TCA CCC AAA TAG 5′

are thus synthesized at the end of the spacer arm.

These various analytical supports are then used to determine targets having a sequence complementary to the probes synthesized on the substrate, these targets comprising, in the 5° position, a fluorescent label consisting either of Cy3 or of Cy5. The fluorescence intensity is then determined for each of the substrates. The results obtained are illustrated in FIG. 3, which represents the fluorescence intensity as a function of the thickness of the layer of SiO₂ (in nm) for Cy3 and for Cy5.

In this figure, it is seen that, for an oxide thickness of 100 nm, a maximizing of the signals emitted by Cy3 and Cy5 is obtained. On the other hand, for a layer of 190 nm, a minimizing of the signals emitted by Cy3 and Cy5 is obtained.

For a layer thickness of 475 nm, a maximizing of the signal emitted by Cy3 and a minimizing of the signal emitted by Cy5 are observed.

For a layer thickness of 545 nm, a maximizing of Cy5 and a minimizing of Cy3 are obtained.

The experimental results obtained are therefore clearly in agreement with the simulations produced by calculation in FIG. 2.

### EXAMPLE 2

In this example, a multilayer stacking is used in order to equilibrate the fluorescence radiations of the fluorophores Cy3 and Cy5. The stacking is produced by depositing, on a glass, the following sequence of thin layers with high refractive index (n=1.836) and with low refractive index (n=1.45):
an 80 nm-thick layer of HfO₂ (high refractive index, n=1.836),
a 102 nm-thick layer of SiO₂ (low refractive index, n=1.45),
an 80 nm-thick layer of HfO₂ (n=1.836),
a 102 nm-thick layer of SiO₂ (n=1.45),
an 80 nm-thick layer of HfO₂ (n=1.836), and
a 108 nm-thick layer of SiO₂ (n=1.45).

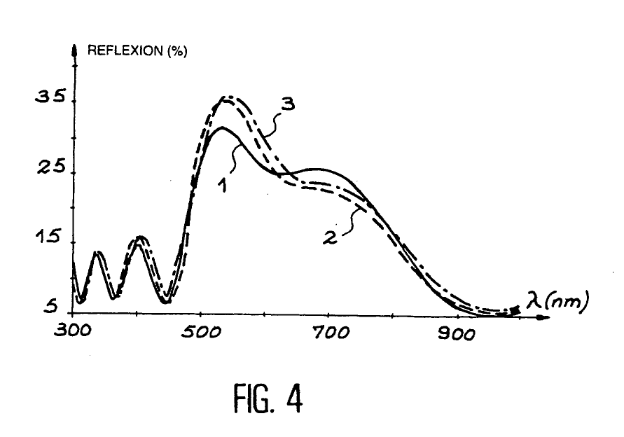

The theoretical and measured responses of this stacking are given in FIG. 4, which illustrates the evolution of the optical properties of reflexion (in %) as a function of the wavelength λ (in nm). Curve 1 refers to the theoretical values and curves 2 and 3 refer to three experimental tests, curve 2 illustrating two tests for which the results are too close to produce the two curves.

In view of this figure, it is noted that the experimental results are clearly in agreement with the simulations produced by calculation.

This stacking can be used to detect the mutation of a gene involved in the synthesis of emerin, a protein involved in triggering Eymery-Dreifuss muscular dystrophy. This stacking is optimized for the fluorophores Cy3 and Cy5.

Cy3: excitation wavelengths of 550 nm and emission wavelengths of approximately 580 nm.

Cy5: excitation wavelengths of 650 nm and emission wavelengths of 680 nm.

Under the reading conditions of the “General Scanning” scanner (numerical aperture of 0.75), theoretical fluorescences of 0.27 (arbitrary unit) are obtained for the two fluorophores Cy3 and Cy5. This makes it possible to equilibrate the emissions of the two fluorophores for the particular case of reading with the “General Scanning” scanner.

### REFERENCES CITED

[1] Médecine/Sciences, Vol. 13, No. 11, 1997, pp. 1317-1324

[2] U.S. Pat. No. 5,578,832

[3] U.S. Pat. No. 5,646,411

[4] Edward H. Hellen and Daniel Axelrod, J. Opt. Soc. Am. B, Vol. 4, No. 3, March 1987, pp. 337 to 350

[5] WO 94/22889.

1-13. (canceled)

14. A method for determining an analyte, comprising the steps of:
depositing the analyte on a support carrying a plurality of ligands, said analyte being labeled by at least a first and a second fluorescent label;
incubating said support under conditions allowing the binding of said analyte with said ligands;
applying an excitatory field to each of the fluorescent labels;
enabling balancing of fluorescent signals emitted by the first and the second fluorescent labels in response to said excitatory field by providing said support comprising:
a substrate,
at least one layer of a material coating the substrate said at least one layer being transparent to the excitation and emission wavelengths and producing an increase or a decrease of the excitatory field for at least one of the first and the second fluorescent labels, said plurality of ligands being attached to said at least one layer; and
detecting the fluorescent signals emitted by the first and second fluorescent labels thereby identifying ligands with which the analyte is bound.

15. The method of claim 14, wherein said depositing comprises depositing the analyte on a support carry ligands comprising DNA or RNA probes.

16. The method of claim 14, wherein said providing said support comprises providing on the substrate at least one layer of material comprising at least one of a refractory oxide, a metal fluoride, or a silicon oxynitride.

17. The method of claim 16, further comprising providing said refractory oxide selected from a group consisting of TiO₂, HfO₂, Ta₂O₅, SiO₂, and ZrO₂.

18. The method of claim 16, further comprising providing said metal fluoride selected from a group consisting of YF₃ and MgF₂.

19. The method of claim 14, wherein said providing said support comprises providing said substrate as at least one of a semi-conductor material, a glass, or a plastic material.

20. The method of claim 19, wherein said providing said support comprises providing said substrate made of silicon.

21. The method of claim 14, wherein said depositing comprises depositing said analyte labeled with Cy₃ and Cy₅, and said providing said support comprises providing a layer of SiO₂ having a thickness of approximately 100 to 1000 nm.

22. The method of claim 14, wherein said enabling comprises providing said support with said at least one layer of material constitutes an optical filter.
